(19) United States
(12) Patent Application Publication
(10) Pub. No.: US 2024/0074813 A1
(43) Pub. Date: Mar. 7, 2024

(54) MEDICAL IMAGING SYSTEM AND METHOD

(71) Applicant: CANON MEDICAL SYSTEMS CORPORATION, Otawara-shi (JP)

(72) Inventor: Steven Alexander REYNOLDS, Edinburgh (GB)

(73) Assignee: CANON MEDICAL SYSTEMS CORPORATION, Otawara-shi (JP)

(21) Appl. No.: 17/929,843

(22) Filed: Sep. 6, 2022

(51) Int. Cl. A61B 34/20

(52) U.S. Cl. CPC A61B 34/20; A61B 2090/365

(57) ABSTRACT

A medical imaging system comprises a head mount display device through which a user can view a patient or an image of the patient, a first tracking system configured to determine a position and orientation of a medical device, a second tracking system configured to determine a position and orientation of the head mount display device, at least one marker whose position and orientation is detectable by the second tracking system and that has a known or determinable position and orientation relative to a frame of reference of the first tracking system, or whose position and orientation is detectable by the first tracking system and that has a known or determinable position and orientation relative to the head mount display device and processing circuitry configured to register the frame of reference of the first tracking system and a frame of reference of the head mount display device using the position and orientation of the at least one marker and display at least one of: a representation of the medical device using the head mount display device, wherein the representation of the medical device is aligned relative to a view or image of the patient based on the registration; and a medical image and a representation of the medical device using the head mount display device, wherein the medical image and the representation of the medical device are aligned based on the registration.

### FIELD

The present invention relates to a medical imaging system and method, for example a method of displaying a medical image and/or a representation of a medical device using a head mount display device.

### BACKGROUND

In medical imaging procedures, live image data may be displayed on a display screen. This may require an operator to switch between looking at a patient or other subject and the display screen, which may not be ergonomic and/or comfortable for the operator.

An augmented reality (AR) or virtual reality (VR) headset may be used during a medical imaging procedure to display the live image data. In such medical imaging procedures, a measurement probe may be used to obtain the image data. Additionally, a medical device may be used to perform a medical procedure. For example, in needle guided biopsy procedures, a needle may be used to obtain tissue samples from a patient or other subject. The needle is inserted into tissue and is guided using the measurement probe.

The headset may be used to display the image data relative to a position of the measurement probe and/or the needle. The measurement probe and/or the needle may be optically tracked. However, the headset, measurement probe and/or needle move independently of each other, which may make it difficult to align the live image data displayed by the headset with the position of the measurement probe and/or the needle. Additionally, the measurement probe and/or the needle may be used outside of a field of view of the headset. Optical tracking of the measurement probe and/or needle may result in the needle biopsy being performed with reduced accuracy or precision and/or speed.

### DESCRIPTION

Embodiments are now described by way of non-limiting example with reference to the accompanying drawings in which:

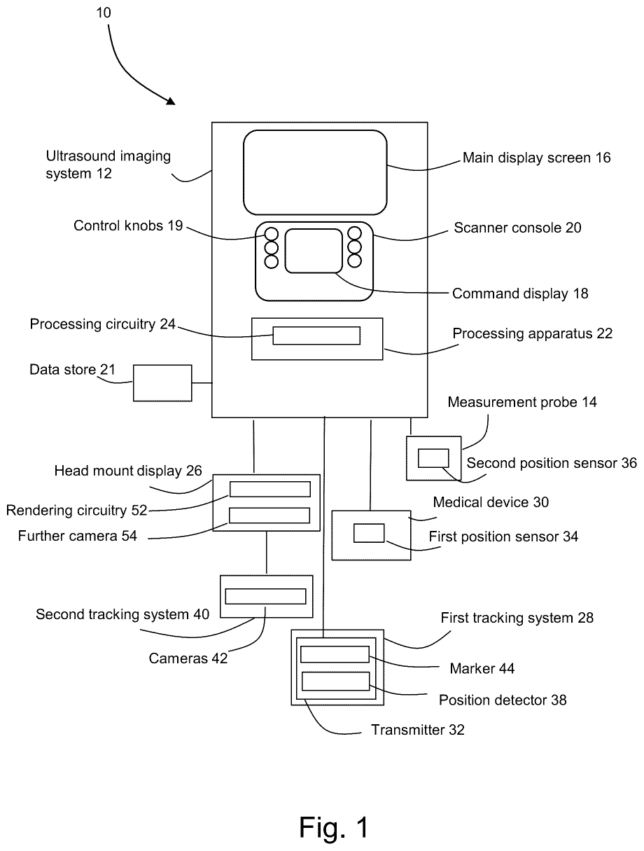

FIG. 1 is a schematic illustration of a medical imaging system in accordance with an embodiment;

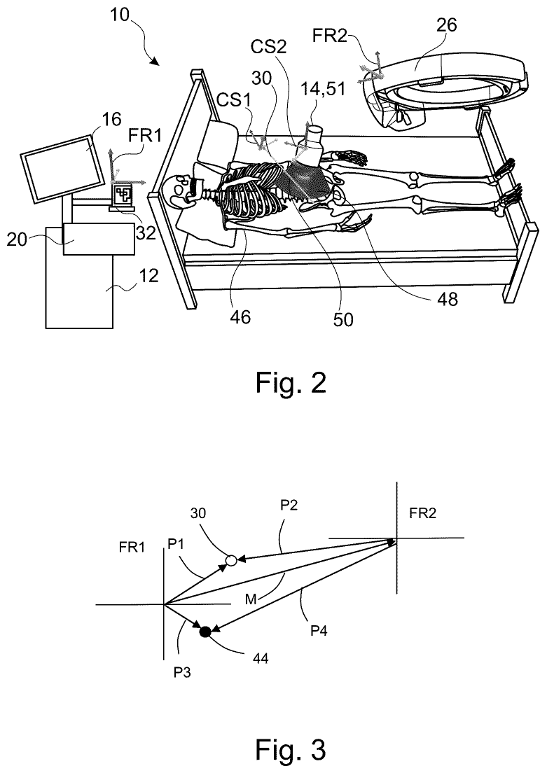

FIG. 2 is a schematic illustration of the system of FIG. 1 in use with a patient;

FIG. 3 is a schematic illustration of a transform between a frame of reference of a first tracking system and a frame of reference of a second tracking system of the system of FIG. 1;

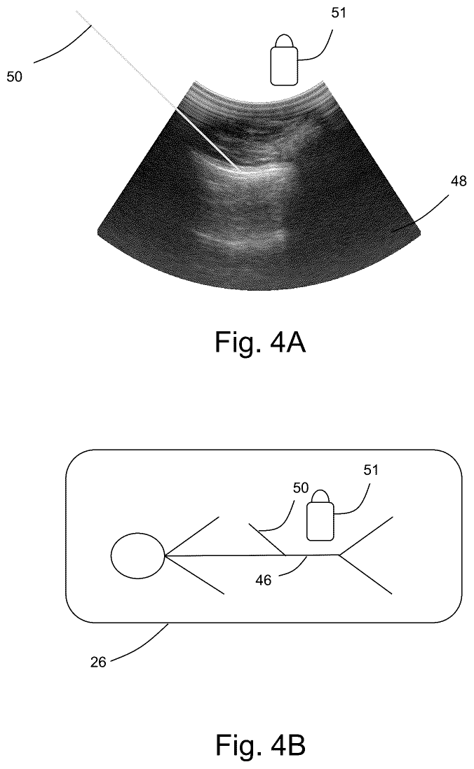

FIG. 4A is an illustration of a medical image and a representation of a medical device of the system of FIG. 1;

FIG. 4B is a schematic illustration of a user's view or image of a patient using a head mount display of the system of FIG. 1;

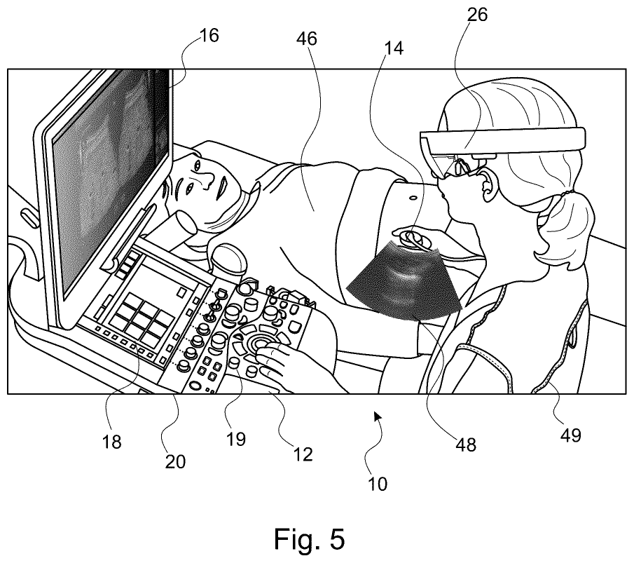

FIG. 5 is another schematic illustration of the system of FIG. 1 in use with a patient;

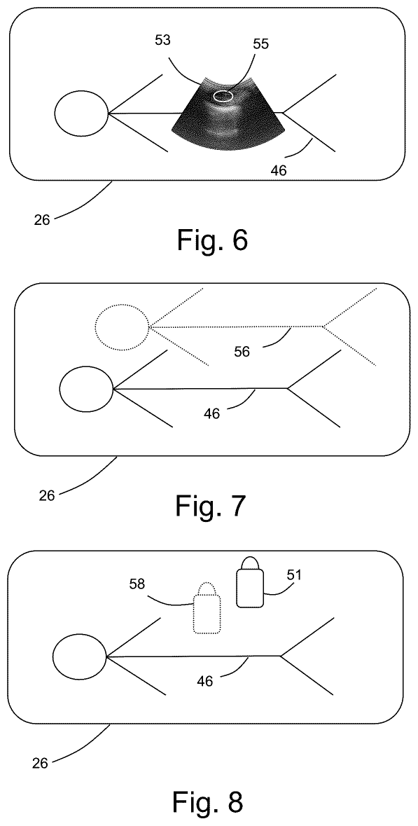

FIG. 6 is a schematic illustration of a user's view or image of a patient using the head mount display of the system of FIG. 1;

FIG. 7 is another schematic illustration of the user's view or image of a patient using the head mount display of the system of FIG. 1;

FIG. 8 is another schematic illustration of the user's view or image of the patient using a head mount display of the system of FIG. 1; and

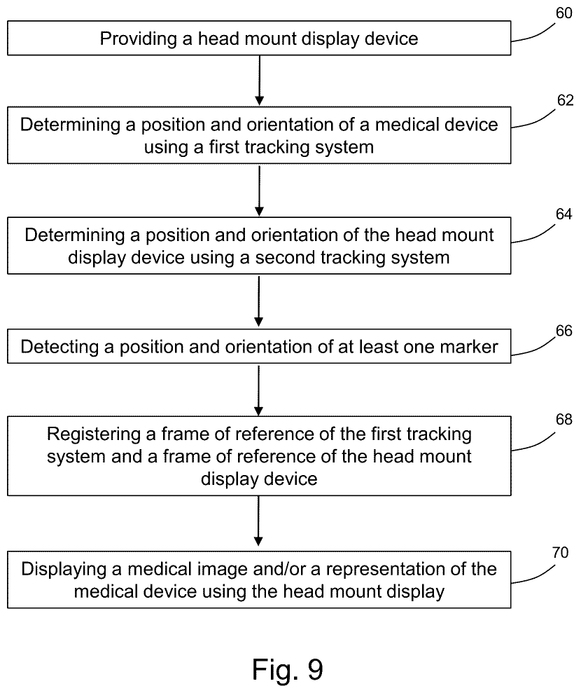

FIG. 9 is a flow chart illustrating in overview a medical imaging method of an embodiment.

Certain embodiments provide a medical imaging system comprising a head mount display device through which a user can view a patient or an image of the patient; a first tracking system configured to determine a position and orientation of a medical device; a second tracking system configured to determine a position and orientation of the head mount display device; at least one marker whose position and orientation is detectable by the second tracking system and that has a known or determinable position and orientation relative to a frame of reference of the first tracking system, or whose position and orientation is detectable by the first tracking system and that has a known or determinable position and orientation relative to the head mount display device; and processing circuitry configured to: register the frame of reference of the first tracking system and a frame of reference of the head mount display device using the position and orientation of the at least one marker; and display at least one of: a representation of the medical device using the head mount display device, wherein the representation of the medical device is aligned relative to a view or image of the patient based on the registration; and a medical image and a representation of the medical device using the head mount display device, wherein the medical image and the representation of the medical device are aligned based on the registration.

Certain embodiments provide a medical imaging method comprising providing a head mount display device through which a user can view a patient or an image of the patient; determining a position and orientation of a medical device using a first tracking system; determining a position and orientation of the head mount display device using a second tracking system; detecting a position and orientation of at least one marker by the second tracking system the at least one marker having a known or determinable position and orientation relative to a frame of reference of the first tracking system, or detecting a position and orientation of the at least one marker by the first tracking system, the at least one marker having a known or determinable position and orientation relative to the head mount display device; registering, using processing circuitry and the position and orientation of the at least one marker, the frame of reference of the first tracking system and a frame of reference of the head mount display device; and displaying, using the processing circuitry and the head mount display device, at least one of: a representation of the medical device that is aligned relative to a view or image of the patient based on the registration; and a medical image and a representation of the medical device, wherein the medical image and the representation of the medical device are aligned based on the registration.

A medical imaging system 10 according to an embodiment is illustrated schematically in FIG. 1. In the present embodiment, the system 10 comprises an ultrasound imaging system 12. In other embodiments, the system may comprise a computer tomography (CT) system, X-ray imaging system or other medical imaging system.

In the present embodiment, the ultrasound imaging system 12 is configured to acquire ultrasound data from an ultrasound scan and to process the ultrasound data to obtain an ultrasound image. The ultrasound imaging system 12 comprises a measurement probe 14. Any suitable type of ultrasound imaging system 12 and measurement probe 14 may be used. The measurement probe 14 may also be referred to as a probe, ultrasound probe or measurement apparatus.

The ultrasound imaging system 12 comprises a main display screen 16 for displaying a main ultrasound image. The ultrasound machine 12 further comprises a scanner console 20. The scanner console 20 comprises a control screen 18 for displaying control information and input devices comprising various control knobs 19. The input devices may further comprise a computer keyboard, a mouse or a trackball (not shown). The control knobs 19 and/or other input devices may be used to adjust values for a plurality of hardware and software parameters. In the present embodiment, the control screen 18 is a touch screen, which is both a display device and a user input device. Further embodiments may comprise a control screen 18, display screen or main display screen 16 that does not form part of the ultrasound machine 12.

The ultrasound machine 12 comprises a data store 21. The ultrasound imaging system 12 comprises a processing apparatus 22 for processing of data, including image data. Image data may also be referred to as ultrasound data. The processing apparatus 22 comprises a Central Processing Unit (CPU) and Graphical Processing Unit (GPU). The processing apparatus 22 includes processing circuitry 24. The processing circuitry 24 may be implemented in the CPU, in the GPU, or in a combination of the CPU and the GPU.

In the present embodiment, the processing circuitry is implemented in the CPU and/or GPU of processing apparatus 22 by means of a computer program having computer-readable instructions that are executable to perform one or more operations of the system 10. However, in other embodiments the processing circuitry may be implemented in software, hardware or any suitable combination of hardware and software. In some embodiments, the various circuitries may be implemented as one or more ASICs (application specific integrated circuits) or FPGAs (field programmable gate arrays).

In alternative embodiments, the processing apparatus 22 comprising the processing circuitry 24 may be part of any suitable medical imaging apparatus (for example a CT system or X-ray imaging system) or image processing apparatus (for example, a PC or workstation). The processing apparatus 22 may be configured to process any appropriate modality of imaging data.

The processing apparatus 22 also includes a hard drive and other components including RAM, ROM, a data bus, an operating system including various device drivers, and hardware devices including a graphics card. Such components are not shown in FIG. 1 for clarity.

The system 10 comprises a head mount display device 26 through which a user can view a patient or other subject or an image of the patient or other subject. The following description will refer to the patient only. However, any of the features disclosed herein may also be applicable to another subject or person. The head mount display device 26 may be provided in the form of an augmented reality (AR) or virtual reality (VR) headset or other wearable head set. The head mount display 26 may also be referred to as a headset.

The system 10 comprises a first tracking system 28. The first tracking system is configured to determine a position and orientation of a medical device 30. The medical device 30 may also be referred to as an intervention device. The medical device 30 may be configured to be inserted into the body of the patient. For example, the medical device may be configured to be inserted into a body cavity, tissue and/or an organ of the patient. In the present embodiment, the medical device 30 is provided in the form of a biopsy needle that may be inserted into the body of the patient to perform a biopsy procedure. In other embodiments, the medical device may be provided in the form of a catheter, an ablation device, an endoscopic device, an endo-cavity probe or any other suitable medical device.

In the present embodiment, the first tracking system 28 is provided in the form of a magnetic tracking system. The first tracking system 28 may also be referred to as electromagnetic tracking system, for example a driveBAY™ or trakSTAR™ electromagnetic tracking system as produced by Ascension Technology Corporation.

The first tracking system 28 comprises a transmitter 32. The transmitter 32 may also be referred to as a generator or magnetic field generator. The transmitter 32 is configured to generate and transmit a magnetic field, such as a DC or AC magnetic field. The first tracking system 28 comprises a first position sensor 34. The first position sensor 34 may be part of the medical device 30. For example, the first position sensor 34 is embedded in the medical device 30. The first position sensor 34 is configured to detect the magnetic field generated by the transmitter 32. For example, the first position sensor 34 is provided in the form of a receiver, in which a voltage may be induced by the magnetic field. Based on a strength and/or direction of the detected magnetic field, the first tracking system 28 is configured to determine the position and orientation of the first position sensor 34 and therefore, the medical device 30.

The first tracking system 28 may comprise a second position sensor 36. The first tracking system 28 is configured to determine a position and orientation of the second position sensor 36. In the present embodiment, the second position sensor 36 is part of the measurement probe 14. For example, the second position sensor 36 is embedded in the measurement probe 14. The second position sensor 36 is configured to detect the magnetic field generated by the transmitter 32. For example, the second position sensor 36 is provided in the form of a receiver, in which a voltage may be induced by the magnetic field. Based on a strength and/or direction of the detected magnetic field, the first tracking system 28 is configured to determine the position and orientation of the second position sensor 36 and therefore, the measurement probe 14.

In alternative embodiments, the first tracking system comprises at least one of or a combination of an inertial tracking system, a Wi-Fi-based positioning system, Bluetooth-based positioning system, time of flight-based tracking system, acoustic tracking and/or an ultrasonic tracking system.

The first tracking system 28 comprises a position detector 38. The position detector 38 may also be referred to as an additional or further position system. The position detector 38 is configured to detect movement of a part of the first tracking system 28. For example, the part of the first tracking system 28 comprises the transmitter 32. In the present embodiment, the position detector 38 is part of the transmitter 32. The position detector 38 is configured to detect movement of the transmitter 32. The position detector 38 may be implemented as an optical or inertial sensor. The inertial sensor may comprise a motion sensor, such as accelerometer, and/or a rotation sensor, such as gyroscope. The optical sensor may be implemented as an image sensor, a camera or other optical sensor. The optical sensor may be part of a computer vision system, such as an object detection system. In other embodiments, the position detector may be arranged to be separate from the transmitter.

The system 10 comprises a second tracking system 40. The second tracking system 40 may also be referred to as a further tracking system. The second tracking system 40 is configured to determine a position and orientation of the head mount display 26. The second tracking system 40 is provided in the form of an optical tracking system, which uses visible light, infrared light or other non-visible light. The second tracking system 40 may also be referred to as an optical system. The second tracking system 40 comprises one or more cameras 42 or other optical sensors. In some embodiments, the cameras or other optical sensors 42 are part of the head mounted display 26. In other embodiments, the cameras or other optical sensors 42 are arranged separately from the head mount display 26. For example, the cameras or other optical sensors 42 may be arranged around the head mount display 26. Other suitable arrangements of the cameras or other optical sensors 42 are possible as long as the position and orientation of the head mounted display 26 can be detected by the cameras or other optical sensors 42. In some embodiments, the cameras or other optical sensors 42 are implemented as infrared cameras or sensors or other non-visible light cameras or sensors.

The system 10 comprises at least one marker 44. In the present embodiment, the marker 44 is implemented as an optical marker. The marker 44 may comprise a pattern and/or colour that is detectable by the second tracking system 40 and/or that distinguishes the marker 44 from its surroundings. The pattern may comprise an arrangement of one or more geometric shapes, such one or more circles, triangles, rectangles, squares and/or any other suitable geometric shapes. The arrangement of the geometric shapes may be regular or irregular. In some embodiments, the marker 44 may be provided in the form of a square marker, rectangular marker, circular marker, Quick Response (QR) code, MaxiCode, and/or any other suitable optical marker. A position and orientation of the marker 44 is detectable by the second tracking system 40. For example, the cameras or other optical sensors 42 are arranged on the head mount display 26 or around the head mount display 26 such that the position and orientation of the marker 44 can be detected by the cameras or other optical sensors 42. The position and orientation of the marker 44 is known or determinable relative to a frame of reference of the first tracking system 28. The frame of reference of the first tracking system 28 may also be referred to as the frame of reference of the magnetic field. For example, in the present embodiment the marker 44 is attached to the transmitter 32. In other embodiments, the marker may be located at the same position as the first or second position sensors. For example, in such embodiments, the marker may be attached to the medical device or the measurement probe.

FIG. 2 schematically illustrates the system 10 in use with a patient 46. The frame of reference of the first tracking system 28 has three dimensions and is indicated in FIG. 2 by the coordinate system labelled FR1. The first tracking system 28 is configured to determine the position and orientation of the medical device 30 relative to the frame of reference FR1 of the first tracking system 28.

A frame of reference of the head mount display device 26 has three dimensions and is indicated in FIG. 2 by the coordinate system labelled FR2. The second tracking system is configured to determine a position and orientation of the head mount display device 26 relative to the frame of reference FR2 of the second tracking system 40. Additionally, the second tracking system 40 is configured to determine the position and orientation of the head mount display device 26 relative to the marker 44.

In the present embodiment, the processing circuitry 24 is configured to register the frame of reference FR1 of the first tracking system 28 and the frame of reference FR2 of the head mount display device 26 using the position and orientation of the marker 44. This will be described with reference to FIG. 3, which schematically illustrates a transform of coordinates between the frames of reference FR1, FR2 of the first and second tracking systems 28, 40. The position of the medical device 30 in the frame of reference FR1 of the first tracking system 28 may be represented by a vector, such as 4-element vector. The orientation of the medical device 30 in the frame of reference FR1 of the first tracking system 28 may be represented by a set of three orthogonal basis vectors. The orientation, e.g. the three orthogonal basis vectors, of the medical device 30 are indicated by the coordinate system CS1 in FIG. 2. The position and orientation of the medical device 30 relative to the frame of reference FR1 of the first tracking system 28 can be expressed together by a first matrix P1. The first matrix P1 may be a 4×4 homogenous matrix, representing the position and orientation of the medical device 30 in frame of reference FR1 of the first tracking system 28.

The position of the medical device 30 in the frame of reference FR2 of the second tracking system 40 may be represented by a vector, such as 4-element vector. The orientation of the medical device 30 in the frame of reference FR2 of the second tracking system 40 may be represented by a set of three orthogonal basis vectors. The position and orientation of the medical device 30 relative to the frame of reference FR2 of the second tracking system 40 can be expressed together by a second matrix P2. The second matrix P2 may be a 4×4 homogenous matrix, representing the position and orientation of the medical device 30 in frame of reference FR2 of the second tracking system 40.

As described above, the first tracking system 28 is configured to determine the position and orientation of the medical device 30. The position and orientation of the medical device 30 is determined relative to the frame of reference FR1 of the first tracking system 28. As such, the first matrix P1 can be determined by the first tracking system 28. For example, the first matrix P1 may be determined based on data representing the position and orientation of the medical device 30 provided by the first tracking system 28. In order to determine the second matrix P2, a determination of a transform M of coordinates from the frame of reference FR1 of the first tracking system 28 to the frame of reference FR2 of the second tracking system 40 is necessary. The determination of the transform M may also be referred to as a calibration.

The position of the marker 44 in the frame of reference FR1 of the first tracking system 28 may be represented by a vector, such as 4-element vector. The orientation of the marker 44 in the frame of reference FR1 of the first tracking system 28 may be represented by a set of three orthogonal basis vectors. The position and orientation of the marker 44 in the frame of reference FR1 of the first tracking system can be expressed together by a third matrix P3. The third matrix P3 may be a 4×4 homogenous matrix, representing the position and orientation of the marker 44 in frame of reference FR1 of the first tracking system 28.

As described above, the marker 44 is attached to the transmitter 32. The transmitter 32 may be considered as an origin of the frame of reference FR1 of the first tracking system. In FIG. 3, the marker 44 is shown as being positioned at a distance relative to the origin of the frame of reference FR1 of the first tracking system 28 for sake of clarity. In other embodiments, the marker may be located at a distance, e.g. a known or determinable distance, from the origin of the frame of reference of the first tracking system.

As described above, the position and orientation of the marker 44 relative to the frame of reference FR1 of the first tracking system 28 is known or determinable. The position and orientation of the marker 44, e.g. relative to the frame of reference FR2 of the second tracking system 40, is detectable by the second tracking system 40. The position of the marker 44 in the frame of reference FR2 of the second tracking system 40 may be represented by a vector, such as 4-element vector. The orientation of the marker 44 in the frame of reference FR2 of the second tracking system 40 may be represented by a set of three orthogonal basis vectors. The position and orientation of the marker 44 relative to the frame of reference FR2 of the second tracking system 40 is expressed together by a fourth matrix P4. The fourth matrix P4 may be a 4×4 homogenous matrix, representing the position and orientation of the marker 44 relative to frame of reference FR2 of the second tracking system 40.

The position and orientation of the marker 44 relative to the frame of reference FR2 of the second tracking system 40 can be determined through optical recognition of the marker 44 by the second tracking system 40, e.g. using an optical recognition algorithm, an image analyser algorithm, video tracking algorithm and/or any other suitable algorithm. The second matrix P2 may be determined based on data representing the position and orientation of the marker 44 provided by the second tracking system 40.

The processing circuitry 24 is configured to determine the transform M of coordinates from the frame of reference FR1 of the first tracking system 28 to the frame of reference FR2 of the second tracking system 40 based on the third and fourth matrices P3, P4. The transform M may be referred to as a transformation and may be implemented as a matrix. The transform M may be derived from or decomposed into one or more affine or linear transforms, such as translations, rotation, shearing and/or scaling. In some embodiments, the transform can be analytically determined, e.g. using one or more analytical methods. The one or more analytical methods may comprise a global equation solver algorithm, Levenberg-Marquardt algorithm and/or any other suitable algorithm. The processing circuitry 24 is configured to implement the registration of the frame of reference FR1 of the first tracking system 28 and the frame of reference FR2 of the second tracking system 40 according to the transform M. In some embodiments, the marker 44 is permanently attached to the transmitter 32. As such, the transform M may be determined only once, unless the transmitter 32 and/or marker 44 has been moved, as will be described below.

In alternative embodiments, a position and orientation of the marker is detectable by the first tracking system. In such alternative embodiments, the marker has a known or determinable position and orientation relative to the head mount display. The marker may be attached to the transmitter, as described above, or may be separately arranged from the transmitter. In such alternative embodiments, the system may be comprise a further position sensor. The further position sensor may be attached, such a temporarily attached, to the marker, or vice versa. The further position sensor is configured to detect the magnetic field generated by the transmitter. For example, the further position sensor is provided in the form of a receiver, in which a voltage may be induced by the magnetic field. Based on a strength and/or direction of the detected magnetic field, the first tracking system is configured to determine the position of the further position sensor and therefore, the marker. The position and orientation of the marker relative to the frame of reference of the second tracking system can be determined through optical recognition of the marker by the second tracking system, as described above. Once the transform has been determined, the further position sensor may be detached from the marker.

FIGS. 2 and 4A show a medical image 48 and a representation 50 of the medical device 30. The representation 50 of the medical device 30 may comprise a three-dimensional representation 50 of the medical device 30. In the present embodiment, the medical image 48 is provided in the form of an ultrasound image. In the present embodiment, the medical image 48 may be part of live imaging data obtained by the ultrasound imaging system. In other embodiments, the medical image may be part of imaging data obtained by another medical imaging system. For example in such other embodiments, the medical image may be provided in the form of an X-ray image, CT scan, MRI scan or other medical image.

The processing circuitry 24 is configured to display the medical image 48 and the representation 50 of the medical device 30 using the head mount display device 26. For example, the processing circuitry 24 is configured to determine the second matrix P2 based on the transform M and the first matrix P1. The processing circuitry 24 is configured to determine the position and orientation of the medical device 30 relative to the frame of reference FR2 of the second tracking system 40. As such, the medical image 48 and the representation 50 of the medical device 30 are aligned based on the registration. The alignment of the medical image 48 and the representation 50 of the medical device 30 may allow a user to perform medical imaging, such as ultrasound imaging, and/or medical procedures, such as needle biopsy, with an increased accuracy and/or speed and/or more ergonomic and/or comfortable.

FIG. 4B schematically illustrates a user's view or image of the 46 patient using the head mount display 26. In some embodiments, the processing circuitry 24 is configured to display a representation 50 of the medical device 30 using the head mount display 26. In such embodiments, the processing circuitry 24 be configured to only display the representation of the medical device 30. As described above, the processing circuitry 24 is configured to determine the second matrix P2 based on the transform M and the first matrix P1. The processing circuitry 24 is configured to determine the position and orientation of the medical device 30 relative to the frame of reference FR2 of the second tracking system 40. As such, the representation 50 of the medical device 30 is aligned relative to a view or image of the patient 46 based on the registration. The alignment of the representation 50 of the medical device 30 relative to the view or image of the patient 46 based on the registration may allow a user to perform medical procedures, such as needle biopsy, with an increased accuracy and/or speed and/or more ergonomic and/or comfortable.

In some embodiments, the processing circuitry 24 is configured to display the medical image 48 and/or the representation 50 of the medical device 30 and a representation 51 of the measurement probe 14. As described above, the first tracking system 28 is configured to determine the position and orientation of the measurement probe 14. The coordinates of the measurement probe 14 are indicated by the coordinate system CS2 in FIG. 2. The processing circuitry 24 is configured to determine the position and orientation of the measurement probe 14 relative to the frame of reference FR2 of the second tracking system 40 based on the transform M, as described above in relation to FIG. 3. In some embodiments, the medical image 48, the representation 50 of the medical device 30 and the representation 51 of the measurement probe 14 are aligned based on the registration, as shown in FIG. 4A. In some embodiments, the representation 50 of the medical device 30 and the representation 51 of the measurement probe 14 are aligned relative to the view or image of the patient 46 based on the registration, as shown in FIG. 4B.

The processing circuitry 24 may be configured to transfer data representing the medical image 30, the representation 50 of the medical device 30 and/or the representation 51 of the measurement probe 14 and the alignment of the medical image 48 with the representation 50 of the medical device 30 and/or the representation 51 of the measurement probe 14 to the head mount display 26. Alternatively, the processing circuitry 24 may be configured to transfer data representing the representation 50 of the medical device 30 and/or the representation 51 of the measurement probe 14 and the alignment of the representation 50 of the medical device 30 and/or the representation 51 of the measurement probe 14 relative to the view or image of the patient 46 to the head mount display 26. In the present embodiment, the head mount display device 26 comprises rendering circuitry 52, such as AR overlay rendering circuitry. The rendering circuitry 52 is configured to display and overlay the medical image 48, the representation 50 of the medical device 30 and/or the representation 51 of the measurement probe 14 based on the data transferred by the processing circuitry 24. In some embodiments, the rendering circuitry 52 is configured to overlay the medical image 48, the representation 50 of the medical device 30 and/or the representation 51 of the measurement probe 14 on a view or image of the patient 46, as illustrated in FIG. 2. FIG. 2 indicates both the measurement probe 14 and the representation 51 of the measurement probe 14 as displayed by the head mount display 26. In some embodiments, the rendering circuitry 52 is configured to overlay the representation 50 of the medical device 30 and/or the representation 51 of the measurement probe 14 on a view or image of the patient 46, as illustrated in FIG. 48.

FIG. 5 illustrates the system 10 in use with a patient. The medical device 30 has been omitted from FIG. 5 for sake of clarity. As described above, the ultrasound imaging system 12 comprises the main display screen 16 for displaying the main ultrasound image. In some embodiments, the processing circuitry 24 is configured to display a representation of the main display screen 16 using the head mount display 26. For example, one or more medical images displayed by the main display screen 16 are displayed using the head mount display 26. Using the head mount display 26 to display the medical image 48 and/or the representation of the main display screen 16 may make the system 10 more ergonomic and/or comfortable for the user 49. For example, the processing circuitry 24 is configured to display the medical image 48, the representation 50 of the medical device 30, the representation 51 of the measurement probe 14 and/or the representation of the main display screen 16 using the head mount display 26, when the user 49 faces the patient 46, the medical device 30 and/or the measurement probe 14. The processing circuitry 24 is configured to not to display the medical image, the representation 50 of the medical device 30, the representation 51 of the measurement probe 14 and/or the representation of the main display screen 16, when the user 49 does not face the patient 46, medical device 30 and/or measurement probe 14. For example, the processing circuitry 24 is configured to display images of the real world, when the user 49 does not face the patient 46, medical device 30 and/or measurement probe 14.

As described above, the first tracking system 28 comprises the position detector 38 configured to detect movement of the transmitter 32, for example relative to the marker 44. The detected movement of the transmitter 32 may be used to invalidate and/or update the registration of the system 10. For example, when movement of the transmitter 32 has been detected the registration of the frames of reference of the first and second tracking systems 28, 40 may no longer be correct or valid and/or may require updating.

In some embodiments, when movement of the transmitter 32 has been detected, e.g. relative to the marker 44, the processing circuitry 24 is configured to alert the user that an update of the registration is required.

In some embodiments, the processing circuitry 24 is configured to update the registration based on a new position of the transmitter 32. The processing circuitry 24 may be configured to determine a new transform M to update the registration. Determining the new transform may comprise determining a new third matrix P3. In embodiments, where a difference between the new position of the transmitter 32 and a previous position of the transmitter 32 is known or determinable, the new third matrix P3 and the new transform M can be determined based on the difference between the new position and previous position of the transmitter 32.

In some embodiments, the head mount display device 26 comprises at least one further camera 54, which is illustrated in FIG. 1. The further camera 54 may also be referred to as a headset camera. The further camera 54 is configured to determine a position and/or orientation of the patient 46. The terms “position and/or orientation of the patient” may encompass a position and/or orientation of a part of a patient's body. The part of the patient's body may be a head, limb, torso or other part of the patient's body. The part of the patient's body may comprise one or more organs or other tissue. The position and orientation of a patient may also be referred to as pose of a patient. In some embodiments, the determined patient position and/or orientation is stored with image data obtained by the system 10. The image data may comprise an ultrasound image 53 and data representing a position and/or orientation of the measurement probe 14, for example relative to the frame of reference FR1 of the first tracking system 28, used to obtain the ultrasound image 53. For example, the determined patient position and/or orientation and the obtained image data can be stored in the data store 21 of the system 10.

FIG. 6 schematically illustrates a user's view or image of the 46 patient using the head mount display 26. In some embodiments, the processing circuitry 24 is configured to use the head mount display device 26 to display an ultrasound image 53 from a prior scan aligned with the view or image of the patient 46. This is illustrated in FIG. 6, which illustrates a user's view or image of the 46 patient using the head mount display 26. In some embodiments, the processing circuitry 24 is configured to use the head mount display device 26 to display an anatomical structure or organ 55 that has been segmented in the prior scan. The processing circuitry 24 is configured to use the head mount display device 26 to display the anatomical structure or organ 55 aligned with the view or image of the patient 46, e.g. in a correct anatomical position.

The processing circuitry 24 is configured to determine a position and/or orientation, such as an initial position and/or initial orientation, of the ultrasound image 53 from the prior scan and/or the anatomical structure or organ 55 segmented in the prior scan relative to a view or image of the patient, for example based on the data representing the position and/or orientation of the measurement probe 14 used to obtain the ultrasound image 53 from the prior scan. The ultrasound image 53 from the prior scan and/or the anatomical structure or organ 55 segmented in the prior scan may be aligned with the view or image of the patient 46 based on the registration between the frame of reference FR1 of the first tracking system 28 and the frame of reference FR2 of the second tracking system 40. For example, the processing circuitry 24 is configured to determine a transform M of one or more coordinates relating to the determined patient position and/or orientation from the frame of reference FR2 of the second tracking system 40 to the frame of reference FR1 of the first tracking system 28. The processing circuitry 24 is configured to determine the transform M based on the third and fourth matrices P3, P4, as described above in relation to FIG. 3.

FIG. 7 schematically illustrates another user's view or image of the 46 patient using the head mount display 26. In some embodiments, the stored patient position and/or orientation is used in a further scan of the patient, for example to guide the patient and/or measurement probe 14 into the same position and/or orientation as in the prior scan. The further scan may also be referred to as a follow-up scan. For example, the processing circuitry 24 may be configured to use the head mount display device 26 to display a representation 56 of the stored patient position and/or orientation, which is illustrated in FIG. 7 by the dotted line, in the further scan. Alternatively or additionally, the processing circuitry 24 may be configured to use the head mount display device 26 to display markers and/or guides to guide the positioning and/or orientation of the patient 46 relative to the stored patient position and/or orientation.

In some embodiments, when a change in the patient position and/or orientation is detected by the further camera 54, for example relative to the stored patient position and/or orientation, the processing circuitry 24 is configured to update the alignment of the image 53 of prior scan and/or the anatomical structure or organ 55 segmented in the prior scan with the changed patient position and/or orientation. The processing circuitry 24 is configured to update the alignment of the image 53 of the prior scan and/or the anatomical structure or organ 55 segmented in the prior scan with the changed patient position and/or orientation based on a difference between the stored patient position and/or orientation and the changed patient position and/or orientation. The processing circuitry 24 is configured to update the alignment by updating the registration based on the difference between the stored patient position and/or orientation and the changed patient position and/or orientation. For example, the processing circuitry 24 may be configured to determine a transform M of one or more coordinates relating to the difference between the stored patient position and/or orientation and the changed patient position and/or orientation from the frame of reference FR2 of the second tracking system 40 to the frame of reference FR1 of the first tracking system 28. The processing circuitry 24 may configured to determine the transform M based on the third and fourth matrices P3, P4, as described above in relation to FIG. 3.

FIG. 8 schematically illustrates another user's view or image of the patient using the head mount display 26. In some embodiments, the processing circuitry 24 is configured to use the head mount display device 26 to display markers and/or guides 58 to guide the positioning and/or orientation of the measurement probe 14 relative to the patient 46, for example to align the measurement probe 14 with a position and/or orientation of the measurement probe 14 used to obtain a prior scan. The display markers and/or guides 58 may also be referred to as visual markers and/or guides. In the embodiment shown in FIG. 8, the display markers and/or guides 58 are implemented in the form of a ghost image of the measurement probe 14, which is illustrated by the dotted line. In other embodiments, the display markers and/or guides may be implemented in the form of one or more instructions to the user, such as up, down, left, right or the like, one or more arrows, or other markers and/or guides.

In alternative embodiments, the processing apparatus is configured to use the head mount display device to display markers and/or guides to guide the positioning and/or orientation of a measurement apparatus of the system relative to the patient, for example to align the measurement apparatus with a position of the part used to obtain a prior scan. For example, in such alternative embodiments, the measurement apparatus may comprise an X-ray generator and X-ray detector or other measurement probe or sensor.

FIG. 9 is a flow chart illustrating in overview a medical imaging method of an embodiment. At stage 60 of FIG. 7, the head mount display device 26 is provided. The user can view a patient or an image of the patient through the head mount display device 26.

At stage 62 of FIG. 9, the position and orientation of a medical device 30 is determined using the first tracking system 28.

At stage 64 of FIG. 9, the position and orientation of the head mount display device is determined using the second tracking system 40.

At stage 66 of FIG. 9, the position and orientation of the marker 44 is detected by the second tracking 40. As described above, the marker 44 has a known or determinable position and orientation relative to the frame of reference FR1 of the first tracking system 28. In some embodiment, the position and orientation of the marker 44 is detected by the by the first tracking system 28. In such embodiments, the marker 44 has a known or determinable position and orientation relative to the head mount display device 26.

At stage 68 of FIG. 9, the frame of reference FR1 of the first tracking system 28 and the frame of reference FR2 of the second tracking system 40 is registered using the processing circuitry 24 and the position and orientation of the marker 44, as described above in relation to FIG. 3.

At stage 70 of FIG. 9, the medical image 48 and/or the representation 50 of the medical device 30 is displayed using the processing circuitry 24 and the head mount display device 26. For example, the medical image 48 and the representation 50 of the medical device 30 are aligned based on the registration, as described above in relation to FIG. 2 or the representation 50 of the medical device 30 is aligned relative to a view or image of the patient 46 based on the registration, as described above in relation to FIG. 4B.

Certain embodiments provide a medical imaging system comprising an augmented reality (AR) or virtual reality (VR) headset or other wearable display device through which a user can view a patient or an image of the patient; an ultrasound imaging system including an ultrasound probe; a magnetic tracking system configured to generate a magnetic field and to determine a position of a needle or other medical device using the magnetic field; a further tracking system configured to determine a position of the AR or VR headset or other wearable device; at least one marker whose position is detectable by the further tracking system and that has a known or determinable position relative to the magnetic field, or whose position is detectable by the magnetic tracking system and that has a known or determinable position relative to the AR or VR headset or other wearable device; and a processor that is configured to: register a frame of reference of the magnetic tracking system and a frame of reference of the AR or VR headset or other wearable device using the position of the at least one marker; and display an ultrasound image and a representation of the needle or other medical device using the AR or VR headset or other wearable device, wherein the ultrasound image and the representation of the needle or other medical device are aligned based on the registration.

The ultrasound image and the representation of the needle or other medical device may be overlaid on a view or image of the patient.

The further tracking system may comprise an optical tracking system using visible light or infra-red light or other non-visible light.

The AR or VR headset or other wearable device may comprise at least one camera configured to determine a position and/or orientation of the patient. The determined patient position and/or orientation may be stored with ultrasound data obtained by the ultrasound imaging system.

The processor may be further configured to use the AR or VR headset or other wearable device to display an image from a prior scan aligned with the view or image of the patient. The processor may be further configured to use the AR or VR headset to display markers and/or guides to guide the positioning of the ultrasound probe relative to the patient, for example to align the ultrasound probe with the position of an ultrasound probe used to obtain a prior scan.

Certain embodiments provide a medical imaging system comprising an head mount display device through which a user can view a patient or an image of the patient; an ultrasound imaging system including an ultrasound probe; a first tracking system configured to determine a position of a needle or other medical device; a second tracking system configured to determine a position of the head mount display device; at least one marker whose position is detectable by the second tracking system and that has a known or determinable position relative to a frame of reference of the first tracking system, or whose position is detectable by the first tracking system and that has a known or determinable position relative to the head mount display device; and a processor that is configured to: register the frame of reference of the first tracking system and a frame of reference of the head mount display device using the position of the at least one marker; and display an ultrasound image and a representation of the needle or other medical device using the head mount display device, wherein the ultrasound image and the representation of the needle or other medical device are aligned based on the registration.

Certain embodiments provide a system configured to display live ultrasound imaging data on an augmented reality headset in a registered acquisition position, the system comprising a magnetic field tracking system that is used to determine a position of a probe or intervention device and an optical system that is used to determine a position of the headset, wherein a registration between magnetic field tracking system and the optical system is established using an optical marker at a known position relative to the magnetic field frame of reference.

An additional position system may be used to detect movement of the magnetic field generator. The detected movement may be used to invalidate or update the calibration for the system.

One or more headset cameras may be used to establish or detect the orientation and/or position of the patient within a coordinate frame of reference of the system. One or more of the established or detected patient's coordinates may be used by an ultrasound imaging system and/or the magnetic field tracking system to determine an initial position of a prior scan. The one or more of the established or detected patient's coordinates may be stored with a position of the prior scan. The one or more of the established or detected patient's coordinates from the prior scan may be used to guide the user to reproduce the same position and orientation of the patient in a follow up scan.

Visual markers and guides may be used on the headset to guide a positioning of the probe.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical imaging system comprising:
a head mount display device through which a user can view a patient or an image of the patient;
a first tracking system configured to determine a position and orientation of a medical device;
a second tracking system configured to determine a position and orientation of the head mount display device;
at least one marker whose position and orientation is detectable by the second tracking system and that has a known or determinable position and orientation relative to a frame of reference of the first tracking system, or whose position and orientation is detectable by the first tracking system and that has a known or determinable position and orientation relative to the head mount display device; and
processing circuitry configured to:
register the frame of reference of the first tracking system and a frame of reference of the head mount display device using the position and orientation of the at least one marker; and
display at least one of:
a representation of the medical device using the head mount display device, wherein the representation of the medical device is aligned relative to a view or image of the patient based on the registration; and
a medical image and a representation of the medical device using the head mount display device, wherein the medical image and the representation of the medical device are aligned based on the registration.

2. A system according to claim 1, wherein the medical image and the representation of the medical device are overlaid on a view or image of the patient.

3. A system according to claim 1, wherein the second tracking system comprises an optical tracking system using visible light or infrared light or other non-visible light.

4. A system according to claim 1, wherein the head mount display device comprises at least one camera configured to determine a position and/or orientation of the patient.

5. A system according to claim 4, wherein the determined patient position and/or orientation are stored with image data obtained by the system.

6. A system according to claim 4, wherein the processing circuitry is further configured to use the head mount display device to display an image from a prior scan aligned with the view or image of the patient.

7. A system according to claim 4, wherein the processing circuitry is further configured to use the head mount display device to display markers and/or guides to guide the positioning of a measurement apparatus of the system relative to the patient to align the measurement apparatus of the system with a position of the measurement apparatus used to obtain a prior scan.

8. A system according to claim 1, wherein the first tracking system is configured to determine a position and orientation of a measurement apparatus of the system and the processing circuitry is configured display a representation of the measurement apparatus using the head mount display device, wherein at least one of:
the medical image, the representation of the medical device and the representation of the measurement apparatus are aligned based on the registration; and
the representation of the medical device and the representation of the measurement apparatus are aligned relative to the view or image of the patient based on the registration.

9. A system according to claim 7, wherein the system comprises an ultrasound imaging system and the measurement apparatus comprises a measurement probe.

10. A system according to claim 1, wherein the head mount display device comprises an augmented reality (AR) or virtual reality (VR) headset or other wearable head set.

11. A system according to claim 1, wherein the first tracking system comprises a magnetic tracking system configured to generate a magnetic field and to determine the position and orientation of the medical device using the magnetic field.

12. A system according to claim 1, wherein the first tracking system comprises at least one of:
a) an inertial tracking system;
b) a Wi-Fi based positioning system;
c) Bluetooth-based positioning system;
d) time of flight-based tracking system;
e) acoustic tracking; and
f) an ultrasonic tracking system.

13. A system according to claim 1, wherein the medical imaging system comprises a screen configured to display the medical image and the processing circuitry is configured to display a representation of the screen using the head mount display device.

14. A system according to claim 1, wherein the processing circuitry is configured to display at least one of: the medical image and the representation of the medical device using the head mount display device, when the user faces the medical device.

15. A system according to claim 1, wherein the first tracking system comprises a first position sensor, the first position sensor being part of or comprised in the medical device.

16. A system according to claim 9, wherein the first tracking system comprises a second position sensor, the second position sensor being part of or comprised in the measurement apparatus.

17. A system according to claim 1, wherein the system comprises a position detector configured to detect movement of a part of first tracking system relative to the at least one marker.

18. A system according to claim 1, wherein when movement of the part of the first tracking system relative to the at least one marker has been detected, the processing circuitry is configured to at least one of:
a) alert a user that an update of the registration is required; and
b) update the registration based on a new position of the part of the first tracking system.

19. A system according to claim 1, wherein the medical device comprises at least one of:
a) a needle;
b) a catheter;
c) an ablation device;
d) an endoscopic device; and
e) an endo-cavity probe.

20. A medical imaging method comprising:
providing a head mount display device through which a user can view a patient or an image of the patient;
determining a position and orientation of a medical device using a first tracking system;
determining a position and orientation of the head mount display device using a second tracking system;
detecting a position and orientation of at least one marker by the second tracking system, the at least one marker having a known or determinable position and orientation relative to a frame of reference of the first tracking system, or
detecting a position and orientation of the at least one marker by the first tracking system, the at least one marker having a known or determinable position and orientation relative to the head mount display device;
registering, using processing circuitry and the position and orientation of the at least one marker, the frame of reference of the first tracking system and a frame of reference of the head mount display device; and
displaying, using the processing circuitry and the head mount display device, at least one of:
a representation of the medical device that is aligned relative to a view or image of the patient based on the registration; and
a medical image and a representation of the medical device, wherein the medical image and the representation of the medical device are aligned based on the registration.
